(19) United States
(12) Patent Application Publication
(10) Pub. No.: US 2023/0312687 A1
(43) Pub. Date: Oct. 5, 2023

(54) CELL-FREE ANIMAL COLLAGEN, METHODS OF PRODUCTION AND USES THEREOF

(71) Applicant: ALEPH FARMS LTD., Rehovot (IL)

(72) Inventors: Neta Lavon, Ness Ziona (IL); Natali Molotski, Givataim (IL); Eldar Zehorai, Tel Aviv (IL); Yair Glick, Petah Tikva (IL); Keren Tazat Fireberg, Ramat Gan (IL)

(21) Appl. No.: 18/041,668

(22) PCT Filed: Aug. 22, 2021
(86) PCT No.: PCT/IL2021/051026

§ 371 Date: Feb. 14, 2023

Related U.S. Application Data
(60) Provisional application No. 63/122,957, filed on Dec. 9, 2020.
(60) Provisional application No. 63/069,049, filed on Aug. 23, 2020.

(51) Int. Cl. C07K 14/78

(52) U.S. Cl. CPC C07K 14/78

(57) ABSTRACT

The present invention relates to the field of use of animal cells for producing products containing animal-derived materials in a sustainable manner, preserving natural resources and promoting animal welfare, particularly for production of cell-free collagen and use thereof for forming textile, particularly leather-like textile, food products, particularly cultured meat, cosmetic products, and medical uses.

### FIELD OF THE INVENTION

The present invention relates to the field of use of animal cells for producing products containing animal-derived materials in a sustainable manner, preserving natural resources and promoting animal welfare, particularly for production of cell-free collagen and use thereof for forming textile, particularly leather-like textile, food products, particularly cell cultured meat, cosmetics, and medical uses.

### BACKGROUND OF THE INVENTION

Collagen is the major proteinaceous component of the extracellular matrix of certain animal species, including mammals. The primary role of collagen is to provide a scaffold to support tissues, although a number of other functions have been elucidated for collagen, including roles in cell attachment, cell migration, filtration and morphogenesis. Skin, or animal hide, contains significant amounts of collagen.

“Collagen” is a name referring to a super family of proteins characterized by repeating triplet of amino acids, -(Gly-X-Y)n-, with glycine being about one-third of the amino acid residues in the collagen. X is often proline and Y is often hydroxyproline, though there may be up to 400 possible Gly-X-Y triplets. Different animals may produce different amino acid compositions of the collagen, which may result in different collagen properties. The collagen structure consists of three intertwined peptide chains of differing lengths, forming the collagen triple helices.

During production of extracellular matrix by fibroblast skin cells, triple helix monomers are synthesized and the monomers may self-assemble into a fibrous form. These triple helices are held together by electrostatic interactions including salt bridging, hydrogen bonding, Van der Waals interactions, dipole-dipole forces, polarization forces, hydrophobic interactions, and/or covalent bonding. The triple helices can be bound together in bundles called fibrils, and fibrils can further assemble to create fibers and fiber bundles. Fibrils have a characteristic banded appearance due to the alternated overlap of collagen monomers. Fibrils and fibers typically branch and interact with each other throughout a layer of skin or hide. Variations of the organization or crosslinking of fibrils and fibers may provide strength to the material.

Currently, there are 28 known distinct collagen types. Collagen types are numbered by Roman numerals, and the chains found in each collagen type are identified by Arabic numerals. Detailed descriptions of structure and biological functions of the various different types of naturally occurring collagens are available in the art (e.g., Ayad et al. 1998. The Extracellular Matrix Facts Book, Academic Press, San Diego, CA).

Collagen Type I is the most prevalent form of collagen in mammalian species and is ubiquitously distributed throughout the body in skin, bone, muscle, tendon, and lung. It is the major structural macromolecule present in the extracellular matrix of multicellular organisms and comprises approximately 20% of total protein mass. Type I collagen is a heterotrimeric molecule comprising two α1(I) chains and one α2(I) chain. Other collagen types are less abundant than type I collagen, and exhibit different distribution patterns. For example, type II collagen is the predominant collagen in cartilage and vitreous humor. Type III collagen is found as a major structural component in hollow organs such as large blood vessels, uterus and bowel. It is also found in many other tissues together with type I collagen.

Collagen has been successfully isolated from various regions of the mammalian body in addition to the animal skin or hide. In more recent years, collagen has been harvested from bacteria and yeast using recombinant techniques.

International (PCT) application Publication No. WO 95/031473 discloses a method for producing collagens from a collagen-producing cells in a cell culturing system. Collagen producing cells are cultured in the presence of an agent to inhibit or interfere with collagen crosslinking. The synthesized collagens are removed from the culture with a solution that maintains the viability of the cells in culture so that collagen synthesis and removal is repeated. The collagens produced by this method are useful for biomedical, biotechnology and cosmetic applications.

The beneficial characteristics of collagen has led to its use in tissue engineering and in a variety of biomedical applications, and a vast effort has been made to produce high-quality collagen. Medical and cosmetic applications of collagen include, for example, skin fillers, wound dressing, and guided tissue regeneration.

Collagen scaffolds have been widely used in tissue engineering as they offer low immunogenicity, a porous structure, good permeability, biocompatibility and biodegradability. These scaffold structures serve as templates with specific mechanical and biological properties similar to native extracellular matrix (ECM).

Collagen is also a primary component in many cosmetic formulations due to the fact that it is a natural humectant and moisturizer. Hydrolyzed Collagen is used primarily in hair preparations and skin care products, but can also be found in makeup, shampoos and bath products. Hydrolyzed Collagen may also be used in hair dyes.

Leather is used in a vast variety of applications, including furniture upholstery, clothing, shoes, luggage, handbags and accessories, as well as in automotive applications. The global trade value of leather is estimated at US $100 billion per year and there is a continuing and increasing demand for leather products. However, use of natural leather from slaughtered animals has encountered public rejection due to increased awareness to animal welfare, use of chemicals hazardous to the environment and health issues. New ways to meet the demand for leather materials amenable to large scale production and exhibiting at least equal or superior properties compared to natural leather are thus required.

For example, International (PCT) Application Publication No. WO 2017/003999 discloses methods of using natural or engineered proteins such as collagen to form tanned and/or cross-linked fibers suitable for a wide range of textile manufacturing processes, including non-woven, woven and knitted fabrics. In particular, described are methods of forming collagen fibers formed from cell-cultured materials by forming a solution of collagen, tanning agent and in some variations cross-linker, and shortly thereafter, extruding collagen fibers. Also described are collagen fibers formed by these methods.

International (PCT) Application Publication No. WO 2017/142887 discloses a bio-fabricated material containing a network of cross-linked collagen fibrils. The material is produced by a process of fibrillation of collagen molecules into fibrils, crosslinking the fibrils and lubricating the cross-linked fibrils.

U.S. Application Publication No. 2019/0203000 discloses a bio-fabricated material containing a network of cross-linked collagen fibrils. This material is composed of collagen which is also a major component of natural leather and is produced by a process of fibrillation of collagen molecules into fibrils, crosslinking the fibrils and lubricating the cross-linked fibrils. Unlike natural leathers, this bio-fabricated material exhibits non-anisotropic (not directionally dependent) physical properties, for example, a sheet of bio-fabricated material can have substantially the same elasticity or tensile strength when stretched or stressed in different directions. Unlike natural leather, it has a uniform texture that facilitates uniform uptake of dyes and coatings. Aesthetically, it produces a uniform and consistent grain for ease of manufacturability. It can have substantially identical grain, texture and other aesthetic properties on both sides distinct from natural leather where the grain increases from one side (e.g., distal surface) to the other (proximal inner layers).

In recent years, public awareness of the resources required for growing livestock animal for meat production, and of the livestock animal welfare, has led to a search for other sources of food. It promoted a search for systems, methods and compositions for producing plant-based meat substitutes/analogues as well as cultured meat products (also referred to as cell-cultured meat, cell-based meat, clean meat, cultivated meat and slaughter free meat products). Among others, a challenge in producing meat substitutes and clean meat products is the texture and mouth-feel that fail to replicate those of equivalent slaughtered-meat products. Adding collagen and other ECM matrix components can significantly improve the textural characteristics of these products.

There is a need for large-scale, low-cost methods of producing high-quality collagen that can be used for the production of collagen-comprising compounds, including leather-like textile, food, medical and cosmetic products, at an affordable price.

### SUMMARY OF THE INVENTION

The present invention provides systems and methods for the production of cell-free animal collagen and/or a medium comprising same in a continuous, scalable manner. The systems and methods are based on a cell culture continuously producing soluble collagen that is secreted into the culture medium, thus enabling easy collection of the soluble collagen. The soluble collagen can thereafter be processed to form collagen fibrils and fibers, and the collagen products can be used in medicine or cosmetics, in the cell cultured food industry or can be further bio-fabricated to form leather-like textile. The cell-free collagen of the invention is animal collagen. The present invention provides in some embodiments cell-free collagen produced by non-genetically engineered cells. The present invention further provides in some embodiments cell-free collagen produced by genetically engineered cells modified to increase collagen production and/or secretion. In certain embodiments, the cells are genetically modified in genes involved in collagen types I, II and III synthesis, collagen secretion and/or collagen crosslinking. According to certain embodiments, the cell free collagen of the invention is non-recombinant collagen.

The present invention is based in part on the unexpected discovery that growing collagen-producing cells in the presence of a procollagen peptidase inhibitor significantly increases the concentration of soluble, non-crosslinked collagen in the medium. Without wishing to be bound by any specific theory or mechanism of action, the continuous production of soluble collagen by the cells in the system of the invention may be attributed to the lower feedback repression by processed collagen present in the medium.

The present invention is based in part on the unexpected discovery that bovine stromal cells, particularly stromal cells differentiated from bovine pluripotent stem cells, can continuously produce and secrete soluble collagen, by culturing the cells in a medium containing a procollagen peptidase inhibitor and exchanging the obtained soluble-collagen enriched medium with collagen-free medium containing the procollagen peptidase inhibitor. The cells produce high amount of soluble collagen suitable for large-scale commercial uses.

According to one aspect, the present invention provides a method of producing cell-free animal collagen comprising the steps of:
(i) growing a plurality of collagen producing animal cells in a cell culture comprising culture medium under conditions inducing collagen synthesis, wherein soluble collagen comprising procollagen is secreted to the culture medium;
(ii) inhibiting or reducing of the cleavage of at least one propeptide from the procollagen N- or C-terminus, thereby obtaining a soluble collagen-enriched medium; and
(iii) collecting the soluble collagen enriched medium while maintaining the cell culture.

According to certain embodiments, the conditions inducing collagen synthesis comprise growing the cells in a culture medium comprising at least one agent inducing collagen synthesis.

According to some embodiments, the at least one agent inducing collagen synthesis is selected from the group consisting of iron, insulin, ascorbic acid or a salt thereof, and any combination thereof. Each possibility represents a separate embodiment of the invention. According to certain exemplary embodiments, the agent inducing collagen synthesis is ascorbic acid or a salt thereof.

According to some embodiments, inhibiting or reducing the cleavage of the at least one propeptide from the procollagen N- or C-terminus comprises supplementing the medium with at least one agent selected from a small molecule, an antibody, a protein, a peptide and an siRNA.

According to some embodiments, the at least one agent inhibits or reduces the activity of at least one of Procollagen C-endopeptidase enhancer (PCOLCE) and PCOLCE2. Each possibility represents a separate embodiment of the present invention.

According to some embodiments, the at least one agent inhibits or reduces the activity of at least one procollagen peptidase.

According to some embodiments, the procollagen peptidase is selected from the group consisting of procollagen N-peptidase and procollagen C-peptidase. Each possibility represents a separate embodiment of the invention.

According to certain embodiments, the collagen peptidase is a matrix metalloprotease (MMP). According to certain embodiments, the MMP is selected from the group consisting of bone morphogenetic protein-1 (BMP1), Meprin α, Meprin β, ADAMTS2, ADAMTS3, ADAMTS14, TLL1, and TLL2. Each possibility represents a separate embodiment of the invention.

According to some embodiments, the at least one agent is a matrix metalloprotease (MMP) inhibitor.

According to some embodiments, the MMP inhibitor is selected from the group consisting of GM6001, TIMP3, Batimastat (BB-94), Actinonin, UK 383367 and any combination thereof. Each possibility represents a separate embodiment of the invention.

According to certain embodiments, the inhibitor is a C-terminal cleavage inhibitor. According to certain exemplary embodiments, the MMP inhibitor is GM6001.

According to some embodiments, the method comprises inhibiting or reducing the cleavage of propeptides from both the procollagen N- and C-terminus.

According to some embodiments, collecting the soluble collagen enriched medium is performed by continuously removing the soluble collagen enriched medium. According to some embodiments, collecting the soluble collagen enriched medium is performed by intermittently removing the soluble collagen enriched medium According to certain embodiments, removing the soluble collagen-enriched culture medium essentially prevents adherence of said soluble collagen to the cell membrane.

According to some embodiments, the method further comprising furnishing the cell culture with a collagen-free culture medium comprising at least one agent inhibiting the cleavage of at least one propeptide from procollagen N- or C-terminus and at least one agent inducing collagen synthesis by the cells. According to certain embodiments, the medium further comprises at least one agent inhibiting crosslinking of soluble collagen secreted to the medium.

According to certain embodiments, the cell culture is furnished with the collagen-free medium by perfusion.

According to some embodiments, the cells are genetically modified.

According to some embodiments, the cells are genetically modified to enhance collagen synthesis, and/or enhance collagen secretion, and/or inhibit collagen crosslinking.

According to some embodiments, the genetic modification affects procollagen post-translational modifications.

According to some embodiments, the cells are genetically modified to inhibit or reduce the cleavage of at least one propeptide from the procollagen N- or C-terminus.

According to some embodiments, the cells are genetically modified to have reduced activity and/or expression of at least one procollagen peptidase. According to certain embodiments, the procollagen peptidase is procollagen-C-proteinase. According to other embodiments, the procollagen peptidase is procollagen-N-proteinase. According to additional embodiments, the cells are genetically modified to have reduced activity and/or expression of both procollagen-C-proteinase and procollagen-N-proteinase.

According to some embodiments, the cells are genetically modified to reduce the expression and/or activity of at least one Lysyl oxidase (LOX). According to certain embodiments, the at least one Lysyl oxidase is selected from the group consisting of Lox, Lox11, Lox12, Lox13, Lox14 and Lox15.

According to some embodiments, the genetically modified gene is lysyl oxidase (LOX). According to certain embodiments, the expression and/or activity of LOX protein is reduced. According to certain embodiments, the gene encoding for LOX protein is knocked out. According to certain exemplary embodiments the LOX gene is silenced by at least one RNAi Molecule. According to certain exemplary embodiments, the LOX gene is silenced by transforming the cell with LOX-targeted siRNA.

According to certain embodiments, the cells are genetically modified to have enhanced expression and/or activity of at least one protein selected from the group consisting of COL1A1, COL1A2, COL3A1, and any combination thereof.

According to some embodiments, the cells are genetically modified to have increased expression and/or activity of lysyl hydroxylase (LH) and/or Prolyl hydroxylase (PH).

According to some embodiments, the cells are genetically modified to have increased expression and/or activity of Galactosyltransferase. According to certain embodiments, the Galactosyltransferase is collagen beta(1-O)galactosyltransferase 1. According to other embodiments, the Galactosyltransferase is collagen beta(1-O)galactosyltransferase 2.

According to some embodiments, the cells are genetically modified to have increased activity of collagen secretion. According certain embodiments, the cells are genetically modified to have increased expression and/or activity of serpin family H member 1 protein (SERPINH1).

According to some embodiments, the cells are genetically modified to alter the expression of at least one gene involved in the feedback inhibition of collagen synthesis. According to certain embodiments, the cells are genetically modified by blocking procollagen N-terminal peptide (PINP) cleavage and/or blocking the PINP receptors.

According to some embodiments, the cells are genetically modified to alter the expression of at least one gene that contributes to fibrosis. According to certain embodiments, the gene is SMAD4. According to some embodiments, the cells are genetically modified to overexpress SMAD4.

According to some embodiments, the cells are genetically modified to increase N-glycosylation.

According to some embodiments, the genetic modification is at the DNA level. According to certain embodiments, the genetic modification is performed by gene-editing method using artificially engineered nucleases. According to certain embodiments, the artificially engineered nucleases are selected from the group consisting of meganucleases, Zinc finger nucleases (ZFNs), transcription-activator like effector nucleases (TALENs), CRISPR/Cas, CRISPR/Cas homologous and CRISPR/Cas modified systems. According to certain exemplary embodiments, the gene editing is performed by the CRISPR/Cas system.

According to some embodiments, the genetic modification comprises transforming the cells with at least one polynucleotide encoding an RNA interfering (RNAi) molecule targeted to at least one gene involved in collagen crosslinking. According to certain embodiments, the polynucleotide encodes an antisense molecule or siRNA.

According to some embodiments, the genes described herein are genetically modified to be activated constitutively by either inserting a copy of the gene with a constitutive promoter or by overexpressing the gene enhancers by genetic modifications.

According to some embodiments, the method comprises a step of genetically modifying the cells to increase at least one of collagen production and/or increase collagen secretion, and/or reduce collagen crosslinking. Each possibility represents a separate embodiment of the invention. According to certain embodiments, the cell modifications are as described hereinabove.

According to some embodiments, the cells are non-genetically modified cells.

According to some embodiments, the method further comprising a step of inhibiting crosslinking of the soluble collagen within the soluble collagen-enriched culture medium.

According to some embodiments, the inhibition of crosslinking of the soluble collagen comprises at least one of depleting copper ions from the culture medium; replacing the medium with a cooper-free medium; and supplementing the medium with at least one lysyl oxidase inhibitor.

According to certain embodiments, depleting copper ions from the culture medium comprises adding at least one cooper-chelating agent. According to certain exemplary embodiments, the cooper chelating agent is Penicillamine (Cuprimine).

According to certain embodiments, the at least one inhibitor of lysyl oxidase is selected from the group consisting of β-aminopropionitrile (BAPN), β-bromoethylamine, β-a-nitroethylamine, benzylamines, diamine analogs, isoniazid, iproniazid, c/s-diaminocyclohexane, hydrazines, semicarbizides, trans-2-phenylcyclopropylamine hydrochloride (tranylcypromine), 2-chloroethylamine hydrochloride and dithiothreitol. Each possibility represents a separate embodiment of the present invention.

According to certain exemplary embodiments, the at least one inhibitor of lysyl oxidase is β-aminopropionitrile.

According to certain embodiments, the plurality of collagen producing cells are non-proliferative functional cells.

According to some embodiments, the cells are pluripotent stem cells (PSCs).

According to certain embodiments, the cells are pluripotent stem cells (PSCs) derived from collagen-producing non-human animal.

According to some embodiments, the cells are stromal cells. According to certain embodiments, the stromal cells are fibroblast cells.

A significant advantage of the methods of the present invention resides in that there is no need to frequently replace the cells, and same cells can continuously produce collagen for a prolonged time, resulting in significant amounts of collected collagen.

According to some embodiments, maintaining the cell culture comprises growing a plurality of the cells without replacing the cells.

According to certain embodiments, collecting the soluble collagen-enriched medium enables maintaining the cell culture for a production cycle of at least 4 days, at least 5 days, least 6 days, least 7 days, least 8 days, least 9 days, or at least 10 days. According to some embodiments, the cell culture is maintained for a production cycle of at least 20 or 30 days.

According to some embodiments, the plurality of animal cells comprises at least 10⁹ cells. According to certain embodiments, the plurality of animal cells comprises at least 10⁹, 10¹⁰, 10¹¹, 10¹², 10¹³, or at least 10¹⁴ cells.

According to some embodiments, the cells can be reused for an additional cycle of soluble collagen production. According to certain embodiments, the method comprising a step of reseeding the cells in fresh medium for starting a new production cycle of soluble collagen.

According to certain embodiments, the cell culture is a 3-dimensional (3D) suspension culture.

According to some embodiments, the suspension culture comprises microcarriers. According to certain embodiments, the cells are adhered to the microcarriers.

The cells to be used with the methods of the present invention can be obtained from any animal having collagen-producing cells. According to certain embodiments, the cells are collagen-producing mammalian cells. According to some embodiments, the mammalian cells are human cells. According to some embodiments, the mammalian cells are non-human mammalian cells.

According to some embodiments, the collagen-producing mammalian cells are obtained from an ungulate.

According to certain embodiments, the ungulate is selected from the group consisting of a bovine, a buffalo, an ovine, an equine, a pig, a giraffe, a camel, a deer, a hippopotamus, and a rhinoceros. Each possibility represents a separate embodiment of the present invention.

According to certain embodiments, the collagen-producing cells are selected from a non-human animal selected from the group consisting of poultry, aquatic animals, invertebrate and reptiles. Each possibility represents a separate embodiment of the present invention.

According to some embodiments, the non-human animal is known to be used for its skin or hide, including, but not limited to bovine, sheep, horse, crocodile and the like.

According to some embodiments, the non-human-animal is a bovine.

According to some embodiments, the reptile is of the Crocodylidae family.

According to some embodiments, the collagen produced by the methods of the invention is a mammalian collagen. According to certain embodiments, the collagen is a non-human animal collagen. According to other embodiments, the collagen is a human collagen.

According to some embodiments, the collagen is a non-recombinant collagen. According to certain embodiments, the soluble collagen is a non-recombinant collagen, having similar or identical properties with respect to the cell endogenous collagen.

According to certain embodiments, employing the method of the present invention results in the production of at least 100 μg soluble collagen per 10⁶ cells in the cell culture. According to certain embodiments, employing the method of the present invention results in the production of at least 100, 200, 300, 400, 500, 600, 700, or 800 μg soluble collagen per 10⁶ cells in the cell culture. According to some embodiments, employing the method of the present invention results in the production of at least 100, 200, 300, 400, 500, 600, 700, or 800 μg soluble collagen/10⁶ cells/day.

According to some embodiments, the method results in cell-free animal soluble collagen production of at least 100, at least 200, at least 300, at least 400, at least 500 or at least 600 μg per 10⁶ cells per production cycle.

According to some embodiments, the soluble-collagen enriched medium further comprises at least one additional ECM component.

According to certain exemplary embodiments, the at least one additional ECM component is a peptide or a protein. According to certain exemplary embodiments, the at least one additional ECM protein is elastin.

According to certain embodiments, employing the method of the present invention further results in the accumulation of elastin in the medium. According to certain embodiments, employing the method of the present invention further results in the accumulation of elastin in the medium in an amount of at least 20% compared with the amount of collagen produced (w/w). According to certain embodiments, employing the method of the present invention further results in the accumulation of elastin in the medium in an amount of about 20% to 40% of the amount of collagen (w/w).

According to certain embodiments, the method of the present invention further comprises subjecting the soluble collagen present in the collected removed medium to conditions enabling formation of tropocollagen.

According to certain embodiments, the method of the present invention further comprises subjecting the soluble collagen present in the collagen-enriched medium or a soluble collagen obtained therefrom to conditions enabling assembly of said soluble collagen to collagen fibrils.

According to certain embodiments, the formed collagen fibrils are subjected to conditions enabling crosslinking of said collagen fibrils to collagen fibers.

Any method as is known in the art can be used to assemble the soluble collagen to collagen fibrils and to crosslink the collagen fibrils to form fibers.

According to certain embodiments, crosslinking of collagen fibrils is performed by any one of enzymatic crosslinking, chemical crosslinking, physical crosslinking, spinning and any combination thereof. Non-woven or woven fabrics can be produced.

According to certain embodiments, the enzymatic crosslinking comprises subjecting the collagen fibrils to at least one enzyme selected from lysyl oxidase and transglutaminase under conditions enabling catalyzing said collagen fibrils crosslinking by the at least one enzyme.

According to certain embodiments, chemical crosslinking comprises adding tannins to the collagen fibrils.

According to some embodiments, chemical crosslinking comprises adding Hyaluronic acid to the soluble collagen. According to certain embodiments, the Hyaluronic acid is activated to promote collagen crosslinking.

According to certain embodiments, physical crosslinking comprises subjecting the collagen fibrils to UV radiation under conditions enabling crosslinking of said collagen fibrils.

According to certain embodiments, fibril spinning comprises electrospinning, jet-spinning and the like.

The soluble collagen, collagen fibrils and/or collagen fibers produced by the methods of the present invention and/or the medium comprising same may be bio-fabricated for a variety of uses. According to certain embodiments, the produced collagen and/or the medium comprising same is bio-fabricated to form textile, food, cosmetic or medical products. Each possibility represents a separate embodiment of the invention. According to certain currently preferred embodiments, the soluble collagen, collagen fibrils, collagen fibers and/or the medium comprising same produced by the methods of the invention are used for the production of textiles, particularly leather-like textiles. The natural, cell-free collagen fibers of the invention are suitable for use in a variety of textile-production methods, as are currently known and as will be known in the art. According to further currently preferred embodiments, the soluble collagen, collagen fibrils, collagen fibers and/or the medium comprising same produced by the methods of the invention are used in the production of food products, particularly cultured meat products.

According to some embodiments, the soluble collagen produced by methods of the invention comprises at least 50% procollagen. According to certain embodiments, the soluble collagen produced by methods of the invention comprises at least 50%, 60%, 70%, or 80% procollagen. Each possibility represents a separate embodiment of the invention.

The present invention further encompasses the soluble collagen, collagen fibrils, collagen fibers and/or medium comprising same produced by the methods of the invention as described herein.

According to certain aspects, the present invention provides a composition comprising culture medium and soluble collagen, wherein the soluble collagen comprises at least 50% procollagen.

According to certain embodiments, the soluble collagen comprises at least 60%, at least 70%, at least 80%, at least 90% or more procollagen. Each embodiment presents a separate embodiment of the present invention.

According to certain embodiments, the culture medium is an animal cell compatible culture medium. According to certain embodiments, the animal cells are human cells. According to other embodiments, the cells are non-human cells.

According to certain embodiments, the soluble collagen is animal collagen. According to certain embodiments, the soluble collagen is a non-recombinant collagen.

According to certain embodiments, the composition further comprises at least one additional ECM component. According to certain exemplary embodiments, the at least one additional ECM component is elastin. According to certain embodiments, the elastin amount is at least about 20% of the amount of the soluble collagen (w/w). According to certain embodiments, the elastin amount is about 20% to 40% of the amount of soluble collagen (w/w).

According to certain embodiments, the composition is devoid of hydrolyzed collagen.

According to additional aspect, the present invention provides a textile comprising at least 5% of collagen fibers of the invention. According to certain embodiments, the textile has leather-like characteristics.

According to another aspect, the present invention provides a medical material comprising at least 5% of soluble collagen of the invention.

According to some embodiments, the medical material comprises at least 5%, 10%, 15%, or 20% of soluble collagen of the invention.

According to an aspect, the present invention provides a food product comprising at least one of the soluble collagen, collagen fibrils, and/or collagen fibers as described herein.

According to some embodiments, the food product comprises at least 5%, 10%, 15%, or 20% of the collagen of the invention.

According to certain embodiments, the food product is a cell cultured meat product.

According to certain aspects the present invention provides a system for producing soluble-collagen enriched medium, comprising:
(i) a first reservoir comprising collage-producing animal cells in a culture medium under conditions inducing collagen synthesis, the reservoir having an inlet and outlet; and
(ii) delivery means for controlling the culture medium composition.

According to some embodiments, the delivery means are configured to continuously or intermittently control the delivery of a medium into and/or out of the first reservoir.

According to some embodiments, the means for continuously or intermittently control the delivery of the medium comprise at least one pump.

According to some embodiments, the delivery means are for removing the soluble collagen enriched medium and/or adding collagen-free medium.

According to some embodiments, controlling the culture medium composition comprises means for monitoring and/or controlling the medium and/or cell conditions. According to some embodiments, the concentration of the medium ingredients and/or physical conditions are monitored/controlled.

According to some embodiments, the medium ingredients are medium supplements selected from: a small molecule, an antibody, a protein, a peptide, an siRNA and any combination thereof. According to certain embodiments, the medium comprises a procollagen peptidase inhibitor.

According to some embodiments, the physical conditions of the medium comprise said medium pH, temperature, viscosity and the like.

According to some embodiments, the delivery is continuous or intermittent.

According to some embodiments, the system further comprises a medium reservoir. According to certain embodiments, the pump is configured for pumping the medium from the medium reservoir to the first reservoir.

According to some embodiments, the system comprises a collecting reservoir.

According to certain embodiments, the medium delivered into the first reservoir is a fresh culture medium. According to some embodiments, the medium delivered out of the first reservoir is a soluble collagen enriched medium.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a schematic presentation of collagen synthesis process and the intervention points to increase soluble collagen in the growth medium according to the teachings of the present invention. Numbers represent the basic outline of collagen synthesis: After mRNA transcription and cytoplasm translocation, (1): peptide chains are formed on ribosomes along the rough endoplasmic reticulum (RER). The preprocollagen is then released into the lumen of the RER. Thereafter the polypeptide undergoes numerous post-translational modifications in the RER/Golgi apparatus: hydroxylation of proline and lysine residues, and glycosylation. This process is dependent on ascorbic acid as a cofactor. (2): Triple helical structure is formed inside the endoplasmic reticulum. This is called procollagen. (3): Procollagen is transported into the Golgi apparatus, where it is packaged and secreted by exocytosis; (4): Once outside the cell, procollagen N- and C-propeptide are removed by specific endo-proteases and tropocollagen is formed; (5): fibril formation is finally achieved by aggregation and self-assembly of the tropocollagen. (6):

Crosslinking of mature collagen fibrils by lysyl oxidase which links hydroxylysine and lysine residues form collagen fibers. Letters represents the invention intervention points in the collagen synthesis process: (a) Genetic manipulation to increase expression and/or activity of lysyl hydroxylase (LH) and/or of Prolyl hydroxylase (PH); (b) Inhibition of the N- and C-propeptide cleavage by peptidase inhibitors, including, but not limited to, GM6001; (c) Inhibition of mature collagen crosslinking by LOX inhibitor or knockdown of LOX genes.

FIG. 2 shows that inhibition of procollagen N- and C-terminals cleavage by GM6001 (MMP inhibitor) enriches the growth media with soluble collagen. Bovine embryonic fibroblasts cells were cultured in Basal medium (left bar) and Basal medium+50 μM GM6001 (right bar). 72 hours later the growth medium was collected, and soluble collagen was measured (N=6). The amount of soluble collagen in the growth medium increased by more than 2 folds (375 vs 825 μg/10\hat{ }6 cells) in the presence of GM6001

FIG. 3 demonstrates that inhibition of procollagen N- and C-terminals cleavage by GM6001 (MMP inhibitor) extends cell culture lifespan while enriches the growth media with soluble collagen. Cells were cultured in basal medium with 50 μM GM6001. Every 24 hours the growth medium was sampled for soluble collagen quantification. Cell culture remained viable for 12 days while soluble collagen levels in the growth medium continued to be high. Addition of GM6001 to the growth medium enables maintaining the cells in the culture for up to 12 days while enriching the growth medium with soluble collagen.

FIG. 4 demonstrates that continuous collection of soluble collagen from cells enable using of cell culture for several soluble collagen production cycles. Cells were seeded at full confluency and treated for enhancement of soluble collagen production. After first production cycle (P1), soluble-collagen enriched growth medium was collected and quantified for soluble collagen followed by passaging of the cells. After second (P2) and third (P3) production cycle, the medium was collected again and measured for soluble collagen (n=3).

FIG. 5 shows that cells are in non-proliferative functional phase when cultured in basal medium. Cells were cultured in basal medium and their proliferation was measured every 24 hours by presto blue assay (Ex 560 nm and Em 590 nm) for 7 days. Cells were maintained in non-proliferative functional phase for 7 days.

FIGS. 6A-6C shows that Col1A1, Col3A1 and Elastin expression levels increases along the differentiation period. RT-PCR analysis of mRNA level showed an increase of Col1A1 (FIG. 6A), Col3A1 (FIG. 6B) and Elastin (FIG. 6C) expression in the differentiated cells comparing to the bPSCs. The high expression levels remained even when the cells were passaged (P2 vs P3). High expression levels indicated that the cells differentiated from pluripotent cells to cells produce ECM component as collagen and elastin.

FIGS. 7A-7B show that silencing of LOX following by inhibition of procollagen N- and C-terminals cleavage by GM6001 (MMP inhibitor) enriches the growth media with soluble collagen. Bovine stromal cells were seeded and transfected with siRNA sequence against LOX and a scramble sequence as control. (FIG. 7A) RT-PCR analysis of LOX mRNA level shows an 80% reduction of LOX expression by siRNA sequence, relative to the control (n=2). (FIG. 7B) siRNA treated cells were supplemented with basal medium, with or without 50 μM GM6001. Growth medium was sampled, and soluble collagen levels were quantified. Combination of LOX silencing with procollagen N- and C-terminals cleavage inhibition by GM6001 increased soluble collagen levels in the growth medium.

FIG. 8 shows that inhibition of procollagen N- and C-terminals cleavage by GM6001 (MMP inhibitor) enriches the growth media with soluble collagen while decreases insoluble collagen levels. Cells were cultured with basal medium were supplemented with different concentration (0-200 μM) of GM6001. Growth medium was collected, and both soluble and insoluble collagen levels were quantified. Results show correlation between enrichment of the growth medium with soluble collagen and reduction in insoluble collagen levels. Addition of GM6001 to basal medium enables to control the balance between soluble and insoluble collagen in the culture and to enrich the growth medium with soluble collagen.

FIG. 9 is a schematic presentation of continuous production of soluble collagen.

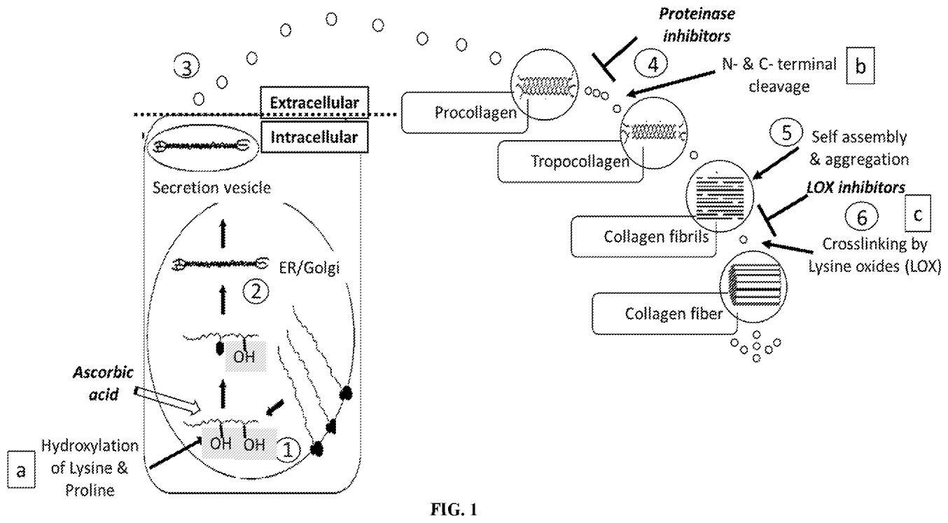

### DETAILED DESCRIPTION OF THE INVENTION

The present invention relates to the field of sustainable manufacturing of products based on animal-derived materials. Particularly, the present invention discloses cost-effective systems and methods for producing macromolecules of animal extracellular matrix (ECM), particularly collagen. The methods of the present invention are characterized by a continuous production of soluble collagen in a cell culture, and are advantageous over hitherto known methods for producing cell-free collagen at least in that (i) same cells are used for prolonged time; (ii) the produced collagen is a native collagen, in certain embodiments of the invention, bovine collagen; or (iii) the collagen in non-fibrillar, soluble collagen. According to some embodiments, the cells are grown in a medium containing collagen peptidase inhibitor. According to some embodiments, production does not involve any genetic modification throughout the production process. According to other embodiments, the cells are genetically modified to reduce the cleavage of the procollagen to prevent the production of tropocollagen. According to additional embodiments, the cells are genetically modified to enhance collagen production and/or inhibit collagen cross-linking. The produced collagen and/or medium comprising same may be bio-fabricated for a variety of uses in the textile, food, cosmetic and pharmaceutic industries. In particular, the produced collagen may be used for textile manufacturing, mainly in manufacturing leather-like textile, for medical uses and in the production of cultured meat.

As used herein, the term “collagen” as used herein refers to any one of the known collagen types, including collagen types I through XX. The term also encompasses procollagens and any collagenous proteins comprising the motif (Gly-X-Y)n where n is an integer. It encompasses molecules of collagen, trimers of collagen molecules, fibrils of collagen, and fibers of collagen fibrils.

As used herein, the term “soluble collagen” refers to any type of collagen secreted from collagen-producing cells into the cell culture medium. According to certain embodiments, the soluble collagen comprises at least 50%, at least 55%, at least 60%, at least 65%, at least 70%, at least 75%, at least 80%, at least 85%, at least 90%, at least 95%, or more, procollagen.

As used herein, the term “procollagen” refers to collagen triple helix monomers comprising globular amino (N)- and/or carboxy (C)-terminal propeptide domains

Subfamilies of collagen include:

Fibril-forming collagens (Collagen Types I, II, II, V, XI, XXIV, and XXVII);

Fibril associated collagens with interrupted triple helices (FACITs) (Collagen Types IX, XII, XIV, XVI, XIX, XX, XXI, XXII). The FACITs do not form fibrils by themselves but they are associated with the surface of collagen fibrils;

Network forming collagens (Collagen Types IV, VIII, X); and

Membrane collagens (Collagen Types XIII, XVII, XXIII, XXV).

Collagen biosynthesis is a complex process comprising inter- and intra-cellular steps. The intracellular steps include gene translation and polypeptide synthesis, particularly synthesis of collagen alpha chains; post-translational modifications, including proline and lysine hydroxylation and glycosylation; and procollagen triple helix formation including proline cis-trans isomerization and stabilization by Heat Shock Protein 47 (HSP47). The soluble procollagen is then secreted to the intercellular space, where it is processed by N- and C-terminal propeptide cleavage to form tropocollagen. Covalent crosslinks are then occurring within and between triple helical collagen molecules to form fibrils, which are assembled to for collagen fibers as part of the ECM.

According to one aspect, the present invention provides a method of producing cell-free animal collagen comprising the steps of:
a) growing a plurality of collagen producing animal cells in a cell culture comprising culture medium under conditions inducing collagen synthesis, wherein soluble collagen comprising procollagen is secreted to the culture medium;
b) inhibiting or reducing of the cleavage of at least one propeptide from the procollagen N- or C-terminus thereby obtaining a soluble collagen-enriched medium; and
c) collecting the soluble collagen enriched medium while maintaining the cell culture.

According to some embodiments, the inhibiting or reducing the cleavage of the at least one propeptide from the procollagen N- or C-terminus comprises supplementing the medium with at least one agent selected from a small molecule, an antibody, a protein, a peptide and an siRNA. Each possibility represents a separate embodiment of the invention.

According to some embodiments, the at least one agent inhibits or reduces the activity of at least one procollagen peptidase.

As used herein the term “procollagen peptidase” refers to enzymes that preform propeptide cleavage and remove the ends of the procollagen molecule to produce tropocollagen.

According to some embodiments, the at least one agent is a matrix metalloprotease (MMP) inhibitor.

According to some embodiments, the MMP inhibitor is selected from the group consisting of GM6001, TIMP3, Batimastat (BB-94), Actinonin, UK 383367 and any combination thereof. Each possibility represents a separate embodiment of the invention.

According to certain embodiments, the inhibitor is a C-terminal cleavage inhibitor. According to certain exemplary embodiments, the MMP inhibitor is GM6001.

GM6001 (also known as Galardin, Ilomastat) is a potent, broad spectrum matrix metalloprotease (MMP) inhibitor. The Ilomastat, having the structure of Formula I, is used herein to inhibit the activity of collagen peptidase.

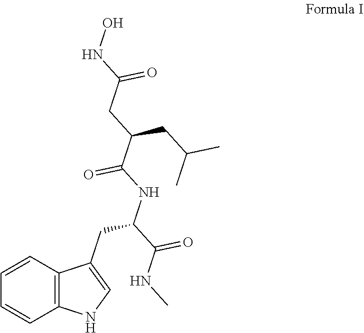 [SMILES: CNC(=O)[C@H](Cc1c[nH]c2ccccc12)NC(=O)[C@@H](CC(=O)NO)CC(C)C]

According to certain embodiments, the cells are pluripotent stem cells. According to these embodiments, the PSCs are typically in a form of PSC aggregates produced by a process as described in International Application Publication No. WO 2020/230138 to the Applicant of the present invention.

According to some embodiments, the cells are genetically modified.

According to some embodiments, the cells are genetically modified to enhance collagen synthesis, and/or enhance collagen secretion, and/or inhibit collagen crosslinking.

According to some embodiments, the genetic modification affects procollagen post-translational modifications.

According to some embodiments, the procollagen peptidase is selected from the group consisting of procollagen N-peptidase and procollagen C-peptidase.

According to some embodiments, the cells are genetically modified to inhibit or reduce the cleavage of at least one propeptide from the procollagen N- or C-terminus.

According to some embodiments, the cells are genetically modified to have reduced activity and/or expression of at least one procollagen peptidase. According to certain embodiments, the procollagen peptidase is procollagen-C-proteinase. According to other embodiments, the procollagen peptidase is procollagen-N-proteinase. According to additional embodiments, the cells are genetically modified to have reduced activity and/or expression of both procollagen-C-proteinase and procollagen-N-proteinase.

According to some embodiments, the procollagen-N-proteinase is selected from the group consisting of ADAM metallopeptidase with thrombospondin type 1 motif 14 (ADAMTS14), ADAM metallopeptidase with thrombospondin type 1 motif 2 (ADAMTS2), and ADAM metallopeptidase with thrombospondin type 1 motif 3 (ADAMTS3). Each possibility represents a separate embodiment of the invention.

The terms “procollagen-N-proteinase”, “procollagen N-peptidase”, and “procollagen N-endopeptidase” are used herein interchangeably and refer to a protease that cleaves the propeptide located at the N-terminus of the procollagen.

Non limiting examples of procollagen-N-proteinases are shown in table 1

TABLE 1
Examples of procollagen-N-proteinases

| tax_id | Org_name | GeneID | Symbol | Description | genomic_nucleotide_accession.version |
|---|---|---|---|---|---|
| 9913 | Bos taurus | 510214 | ADAMTS14 | ADAM | NC_037355.1 |
| | | | | metallopeptidase | |
| | | | | with | |
| | | | | thrombospondin | |
| | | | | type 1 motif 14 | |
| 9913 | Bos taurus | 282401 | ADAMTS2 | ADAM | NC_037334.1 |
| | | | | metallopeptidase | |
| | | | | with | |
| | | | | thrombospondin | |
| | | | | type 1 motif 2 | |
| 9913 | Bos taurus | 534082 | ADAMTS3 | ADAM | NC_037333.1 |
| | | | | metallopeptidase | |
| | | | | with | |
| | | | | thrombospondin | |
| | | | | type 1 motif 3 | |

According to some embodiments, the procollagen-C-proteinase is selected from the group consisting of bone morphogenetic protein 1 (BMP1tolloid like 1 (TLL1), and tolloid like 2 (TLL2). Each possibility represents a separate embodiment of the invention.

The terms “procollagen-C-proteinase”, “procollagen C-peptidase”, and “procollagen C-endopeptidase” are used herein interchangeably and refer to a protease that cleaves the propeptide located at the C-terminus of the procollagen.

Non limiting examples of procollagen-C-proteinases are shown in table 2.

TABLE 2
Examples of procollagen-C-proteinases

| tax_id | Org_name | GeneID | Symbol | Description | genomic_nucleotide_accession.version |
|---|---|---|---|---|---|
| 9913 | Bos taurus | 510360 | BMP1 | bone | NC_037335.1 |
| | | | | morphogenetic | |
| | | | | protein 1 | |
| 9913 | Bos taurus | 504471 | PCOLCE | procollagen C- | NC_037352.1 |
| | | | | endopeptidase | |
| | | | | enhancer | |
| 9913 | Bos taurus | 513618 | PCOLCE2 | procollagen C- | NC_037328.1 |
| | | | | endopeptidase | |
| | | | | enhancer 2 | |
| 9913 | Bos taurus | 616468 | TLL1 | tolloid like 1 | NC_037344.1 |
| 9913 | Bos taurus | 504975 | TLL2 | tolloid like 2 | NC_037353.1 |

The Bone Morphogenetic Protein 1 (BMP1) or “Procollagen C-Endopeptidase” is a metalloprotease that plays key roles in regulating the formation of the extracellular matrix (ECM) via processing of various precursor proteins into mature functional enzymes or structural proteins. An example identifier of BMP1 is UniProtKB/Swiss-Prot: P13497.

According to some embodiments, the at least one agent inhibits or reduces the activity of at least one of Procollagen C-endopeptidase enhancer (PCOLCE) and PCOLCE2. Each possibility represents a separate embodiment of the present invention.

According to certain embodiments, the genetically modified gene is involved in the system of collagen production inhibition through a feedback loop. In some embodiments, the gene involved in this process is procollagen-N-proteinase. In some embodiments, the gene is procollagen-C-proteinase.

According to some embodiments, the cells are genetically modified to enhance collagen synthesis, and/or enhance collagen secretion, and/or inhibit collagen crosslinking.

According to some embodiments, the cells are genetically modified to reduce the expression and/or activity of at least one Lysyl oxidase (LOX).

According to certain embodiments, the at least one Lysyl oxidase is selected from the group consisting of Lox, Lox11, Lox12, Lox13, Lox14 and Lox15. Each possibility represents a separate embodiment of the invention.

Lysyl oxidase (LOX) is an extracellular metalloenzyme which mediates crosslinking of collagen and elastin. Non-limiting examples of Lox proteins include lysyl oxidase (LOX; gene ID 280841), lysyl oxidase like 1 (LOXL1; gene ID 281903), lysyl oxidase like 4 (LOXL4; gene ID 281904), lysyl oxidase like 3 (LOXL2; gene ID 613599), and lysyl oxidase like 2 (LOXL2, gene ID 532684).

According to certain embodiments, the expression and/or activity of Lox protein is reduced. According to certain embodiments, the gene encoding for Lox protein is knocked out. According to certain exemplary embodiments the LOX gene is silenced by at least one RNAi Molecule. According to certain exemplary embodiments, the LOX gene is silenced by transforming the cell with LOX-targeted siRNA. According to certain exemplary embodiments, the siRNA sequence is set forth in SEQ ID NO:1.

According to certain embodiments, the cells are genetically modified in a gene involved in increasing collagen production, including, but not limited to Collagen 1A1, Collagen 1A2, and Collagen 3A1.

According to certain embodiments, the cells are genetically modified in a gene involved in post translational modification of the procollagen. According to certain embodiments, the gene is lysyl hydroxylase (LH). According to some embodiments, the lysyl hydroxylase (LH) is Lysyl hydroxylase-2 (LH2). According to certain embodiments, the lysyl hydroxylase is selected from the group consisting of procollagen-lysine, 2-oxoglutarate 5-dioxygenase 1 (PLOD1), procollagen-lysine, 2-oxoglutarate 5-dioxygenase 2 (PLOD2), and procollagen-lysine, 2-oxoglutarate 5-dioxygenase 3 (PLOD3).

Lysyl hydroxylases (LHs) belong to the large superfamily of 2-oxoglutarate-(aka: alpha-ketoglutarate) dependent oxygenases that requires alpha-ketoglutarate (α-KG) and Fe(II) for activity. These enzymes couple the two-electron oxidation of substrate to the oxidative decarboxylation of α-KG to yield succinate and carbon dioxide. LHs consist of three family members—LH1, LH2 and LH3. While all LHs catalyze the hydroxylation of lysine residues on collagen, only LH2 can modify telopeptidyl lysine residues. Lysine hydroxylation is important in the formation of stable collagen cross-links, that connect collagen molecules and stabilize the extracellular matrix (Devkota et al. SLAS Discov. 2019 April; 24 (4): 484-491).

According to certain embodiments, the cells are genetically modified to have an increased expression and/or activity of Prolyl hydroxylase (PH). According to some embodiments, the Prolyl hydroxylase is selected from the group consisting of prolyl 4-hydroxylase subunit alpha 1 (P4HA1), prolyl 4-hydroxylase subunit alpha 2 (P4HA2), prolyl 4-hydroxylase subunit alpha 3 (P4HA3), prolyl 4-hydroxylase subunit beta (P4HB), prolyl 3-hydroxylase 2 (P3H2), Prolyl 3-hydroxylase-1 (P3H1), and prolyl 3-hydroxylase 3 (P3H3). Each possibility represents a separate embodiment of the invention.

According to some embodiments, the cells are genetically modified to have an increased expression and/or activity of galactosyltransferase. According to certain embodiments the galactosyltransferase is selected from the group consisting of collagen beta(1-O)galactosyltransferase 1 (COLGALT1) and collagen beta(1-O)galactosyltransferase 2 (COLGAT2). Non-limiting example of galactosyltransferase are COLGALT1 (GeneID 5143167) and COLGAT2 (GeneID 789150) of Bos taurus.

According to some embodiments, the cells are genetically modified to have increased activity of collagen secretion. According certain embodiments, the cells are genetically modified to have increased expression and/or activity of serpin family H member 1 protein (SERPINH1). A non-limiting example of SERPINH1 is of the Bos taurus, GeneID 510850.

According to certain embodiments, the method comprises inhibiting of collagen crosslinking.

According to certain embodiments, inhibiting crosslinking of the collagen comprises at least one of depleting copper ions from the culture medium; replacing the medium with a cooper-free medium; and adding at least one inhibitor of lysyl oxidase.

According to certain embodiments, depleting copper ions from the culture medium comprises adding at least one cooper-chelating agent. According to certain exemplary embodiments, the cooper chelating agent is Penicillamine (Cuprimine).

According to some embodiments, the medium comprises at least one inhibitor of lysyl oxidase. According to certain embodiments, the at least one inhibitor of lysyl oxidase is selected from the group consisting of 62 -aminopropionitrile (BAPN), β-bromoethylamine, β-a-nitroethylamine, benzylamines, diamine analogs, isoniazid, iproniazid, c/s-diaminocyclohexane, hydrazines, semicarbizides, trans-2-phenylcyclopropylamine hydrochloride (tranylcypromine), 2-chloroethylamine hydrochloride and dithiothreitol. Each possibility represents a separate embodiment of the present invention.

According to certain exemplary embodiments, the at least one inhibitor of lysyl oxidase is β-aminopropionitrile, a specific an irreversible inhibitor of lysyl oxidase activity.

According to an additional aspect, the present invention provides a method of producing cell-free animal collagen comprising the steps of:
a) growing a plurality of collagen producing animal cells in a cell culture comprising culture medium under conditions inducing collagen synthesis, wherein soluble collagen comprising procollagen is secreted to the cell culture medium, wherein the cells are genetically modified to inhibit or reduce the cleavage of at least one propeptide from the procollagen N- or C-terminus, thereby obtaining a soluble collagen-enriched medium; and
b) collecting the soluble collagen enriched medium while maintaining the cell culture.

According to certain aspects the present invention provides a system for producing soluble collagen, the system comprising cells derived from collagen-producing animal; culture medium supporting growth of the cells, typically comprising at least one procollagen peptidase inhibitor, optionally comprising at least one agent inhibiting crosslinking of soluble collagen to collagen fibrils, further optionally comprising at least one agent enhancing collagen production in said cells; means for supporting continuous growth of said cells and exchange of the culture medium, preferably continuous exchange; and optionally means for collecting the medium comprising the produced soluble collagen, potentially with additional ECM molecules.

According to certain aspects, the present invention provides a system for producing soluble collagen, the system comprising non-genetically modified cells.

According to some embodiments, the collagen is a non-recombinant collagen. According to certain embodiments, the collagen is a non-recombinant collagen, having similar or identical properties with respect to the endogenous collagen of the cell-producing collagen.

According to certain exemplary embodiments, the collagen is in its native form.

According to certain aspects, the present invention provides a system for producing soluble collagen, the system comprising cells genetically modified to decrease crosslinking of secreted collagen in the growth medium.

According to certain aspects, the present invention provides a system for producing soluble collagen, the system comprising cells genetically modified to increase production and secretion of collagen to the growth media as described herein.

According to certain embodiments, the cells are pluripotent cells. According to certain embodiments, the means for exchange of the culture medium comprises means for removing culture medium enriched with soluble collagen secreted from the cells and furnishing said cells with collagen-free culture medium. According to certain embodiments, the means are adapted for continuous exchange of the culture medium. According to certain embodiments, the means for continuous exchange of the culture medium are configured in perfusion mode of said culture.

According to an aspect, the present invention provides a method of producing cell-free collagen comprising the steps of: (a) obtaining a plurality of non-genetically modified animal stromal cells; (b) growing the stromal cells in a cell culture comprising culture medium under conditions inducing collagen synthesis, wherein the medium comprises an inhibitor of collagen peptidase, wherein soluble collagen is secreted to the culture medium, thereby obtaining a soluble collagen-enriched medium; (c) optionally, inhibiting crosslinking of the soluble collagen in the soluble collagen-enriched culture medium; (d) continuously removing said soluble collagen enriched medium, without essentially interfering with the growth of said stromal cells; and (e) collecting the removed soluble collagen-enriched medium.

According to an additional aspect, the present invention provides a method of producing cell-free collagen comprising the steps of: (a) obtaining a plurality of genetically modified animal cells; (b) growing the cells in a cell culture comprising culture medium under conditions inducing collagen synthesis, the culture medium comprises an inhibitor of collagen peptidase, wherein soluble collagen is secreted to the culture medium, thereby obtaining a soluble collagen-enriched medium; (c) optionally inhibiting crosslinking of the soluble collagen in the soluble collagen-enriched culture medium; (d) continuously removing said soluble collagen enriched medium essentially without interfering with the growth of said cells; and (e) collecting the removed soluble collagen-enriched medium.

The extracellular matrix (ECM) of animal issues, particularly mammalian tissues comprise over 1,000 proteins that together provide both mechanical support and signaling functions. One of the main families of ECM proteins are the collagens among which are the fibrillar collagens. Fibrillar collagens are secreted as soluble procollagens and the stabilization of the fibrils is provided by covalent crosslinks.

According to certain embodiments, induction of collagen production within the cells is induced by adding to the cell culture medium at least one of insulin and ascorbic acid or a salt thereof.

According to some embodiments, the method comprises a step of genetically modifying the animal cells, particularly mammalian cells to reduce the cleavage of procollagen in the growing medium.

According to some embodiments, the method comprises a step of genetically modifying the animal cells, particularly mammalian cells to increase collagen production, increase collagen secretion, and/or reduce collagen crosslinking.

According to some embodiments, the method comprises a step of genetically modifying at least one gene involved in increasing collagen production, increasing collagen secretion, and/or reducing collagen crosslinking.

According to some embodiments, the genetic modification increases expression of at least one enzyme involved in collagen production, increasing collagen secretion, and/or reducing collagen crosslinking. In additional embodiments, the genetic modification increases activity of at least one enzyme involved in collagen production, collagen secretion, and/or reducing of collagen crosslinking.

According to certain embodiments, gene modification can enhance collagen production and secretion by modification of genes involved in collagen production, secretion and collagen synthesis feedback inhibition process.

According to certain embodiments, inhibiting crosslinking of the collagen comprises genetic modification of at least one gene involved in procollagen processing and crosslinking.

According to certain embodiments, inhibiting crosslinking of the collagen by blocking procollagen processing comprises modification of the procollagen-N-proteinase and/or procollagen-C proteinases gene.

According to some embodiments, the genetically modified cells produce at least 10%, 20%, 40%, 50%, 60%, 70%, 80%, 90%, or 100% more collagen compared to the corresponding non-genetically modified cells.

According to certain exemplary embodiments, the present invention provides a method of producing cell-free animal collagen comprising the steps of:
(i) obtaining a plurality of collagen producing animal cells;
(ii) growing the cells in a cell culture comprising culture medium under conditions inducing collagen synthesis, wherein soluble collagen is secreted to the culture medium;
(iii) inhibiting or reducing collagen peptidase activity in the collagen-enriched culture medium, thereby obtaining a soluble collagen-enriched medium;
(iv) continuously removing the soluble collagen enriched medium without interfering with the growth of said cells; and
(v) collecting the removed soluble collagen-enriched medium.

According to some embodiments, the step of inhibiting or reducing of the collagen peptidase activity is performed by a matrix metalloprotease (MMP) inhibitor. According to certain exemplary embodiments, the inhibitor is Ilomastat (GM6001).

According to the teachings of the present invention, culture medium enriched with collagen is continuously exchanged with collagen-free culture medium, and the collagen-enriched medium, typically further comprising additional ECM-components is collected. According to certain embodiments, the collagen-producing culture of the present invention is maintained until the collagen concentration within the collected medium reaches a desired concentration.

According to certain embodiments, employing the method of the present invention results in the production of at least 10, at least 50, at least 60, at least 70, at least 80, at least 90 or at least 100 mg collagen per 10⁶ cells per day.

According to some embodiments, employing the method of the present invention results in the production of at least 350 ug soluble collagen per 10⁶ cells in the medium. According to certain embodiments, employing the method of the present invention results in the production of at least 400, 450, 500, 550, 600, 650, 700, 750, 800, 850, 900, 950, or 1000 ug soluble collagen per 10⁶ cells in the medium. According to some embodiments, employing the method of the present invention results in the production of at least 400, 500, 600, 700, 800, 900, or 1000 ug soluble collagen/10⁶ cells/3 days.

According to some embodiments, the method results in cell-free animal soluble collagen production of at least 10, at least 50, at least 100, at least 200 μg, at least 300 μg, at least 400 μg, at least 500 μg, at least 600 μg, at least 700 μg, at least 800 μg, at least 900 μg, or at least 1 mg per 10⁶ cells per production cycle.

The term “production cycle” as used herein refers to a production of soluble collagen from the same batch of cells without a step of passing or replacing the cells. The production cycle may include several replacements of the soluble-collagen enriched medium with a new medium.

According to certain embodiments, employing the method of the present invention results in the accumulation of elastin in the medium. According to certain embodiments, employing the method of the present invention results in the accumulation of elastin in the medium in an amount of at least 10% compared with the amount of collagen (w/w). According to certain embodiments, employing the method of the present invention results in the accumulation of elastin in the medium in an amount of about 10% to 40% of the amount of collagen (w/w). According to certain embodiments, employing the method of the present invention results in the accumulation of elastin in the medium in an amount of about 15% to 35% of the amount of collagen (w/w). According to certain embodiments, employing the method of the present invention results in the accumulation of elastin in the medium in an amount of about 20% to 40% of the amount of collagen (w/w).

According to certain aspects, the present invention provides a composition comprising liquid culture medium and soluble collagen, wherein the soluble collagen comprises at least 50% procollagen.

According to certain embodiments, the soluble collagen comprises at least 60%, at least 65%, at least 70%, at least 75%, at least 80%, at least 85%, at least 90%, at least 95%, or more tropocollagen. Each embodiment presents a separate embodiment of the present invention. According to certain embodiments, the soluble collagen comprises from about 50% to about 95% procollagen. According to some embodiments, the soluble According to some embodiments, the soluble collagen comprises from about 70% to about 85% procollagen.

According to certain embodiment, the composition comprises from about 50 μg/ml to about 150 μg/ml procollagen. According to certain embodiment, the composition comprises from about 75 μg/ml to about 125 μg/ml procollagen. According to some embodiment's, the composition comprises about 100 μg/ml procollagen.

According to certain embodiments, the culture medium is an animal cell compatible culture medium. According to certain embodiments, the animal cells are human cells. According to other embodiments, the cells are non-human cells.

According to certain embodiments, the soluble collagen is animal collagen. According to certain embodiments, the soluble collagen is a non-recombinant collagen.

According to certain embodiments, the composition further comprises at least one additional ECM component. According to certain exemplary embodiments, the at least one additional ECM component is elastin. According to certain embodiments, the elastin amount is at least about 20% of the amount of the soluble collagen (w/w). According to certain embodiments, the elastin amount is about 20% to 40% of the amount of soluble collagen (w/w).

According to certain embodiments, the composition is devoid of hydrolyzed collagen. According to some embodiments, the composition is substantially devoid of hydrolyzed collagen peptides.

### Uses

According to certain embodiments, the soluble collagen is subjected to conditions enabling assembly of said soluble collagen to collagen fibrils and further to collagen fibers.

The term “collagen fibril(s)” as used herein refer to nanofibers composed of tropocollagen (triple helices of collagen molecules). The collagen fibrils of the invention may have diameters ranging from 1 nm and 1 μm. For example, the collagen fibrils of the invention may have an average or individual fibril diameter ranging from 1, 2, 3, 4, 5, 10, 15, 20, 30, 40, 50, 60, 70, 80, 90, 100, 200, 300, 400, 500, 600, 700, 800, 900, or 1,000 nm (1 μm). This range includes all intermediate values and subranges. In some embodiments the collagen fibrils will have diameters and orientations similar to those in the top grain or surface layer of a bovine leather. In other embodiments, the collagen fibrils may have diameters comprising the top grain and those of a corium layer of a conventional leather.

The term “collagen fiber” as used herein refers to a structure composed of collagen fibrils. In certain embodiments, the collagen fibrils are tightly packed. It can vary in diameter from more than 1 μm to more than 10 μm, for example >1, 2, 3, 4, 5, 6, 7, 8, 9, 10, 11, 12 μm or more.

The fibrillar type I and III collagens are derived from the precursor molecules, procollagens (˜450 kDa), which are rod-like, triple-helical molecules (˜300 nm in length) with globular amino (N)- and carboxy (C)-terminal propeptide domains. During its cellular expression and secretion, the procollagens are assembled in the trimeric form and then cleaved at specific N- and C-terminal sites by specific endopeptidases, generating three fragments: the procollagen type I N-terminal propeptide (PINP), type I collagen, and procollagen type I carboxy-terminal propeptide (PICP).

Crosslinking of soluble collagen to fibrils and assembly to fibers can be performed by any method as is known in the art.

Enzymatic Crosslinking: lysyl oxidase/transglutaminases are calcium ion dependent enzymes and are active over a wide range of pH values and temperatures. Being biodegradable, transglutaminases derived from microorganism are benign to cells and can catalyze crosslinking in a concentration dependent manner.

Chemical Crosslinking: Tannic acid (TA) is a hydrolysable plant tannin, that functions as a collagen cross-linking agent through hydrogen-bonding mechanisms and hydrophobic effects. Tannins bind to the collagen proteins in the hide and coat them, causing them to become less water-soluble and more resistant to bacterial attack. The process also causes the hide to become more flexible.

Hyaluronic acid (HA) function as a Collagen cross-linking agent through oxidization of the Hyaluronic acid. The activated HA bind to the Collagen through carboxyl groups.

Physical Crosslinking: combined with photosensitizers such as riboflavin, UV light irradiation is capable of introducing intrafibrillar and interfibrillar carbonyl-based collagen covalent bonds.

Electrospinning: it is a fiber production method which uses electric force to draw charged threads of polymer solutions or polymer melts up to fiber diameters in the order of some hundred nanometers.

The soluble collagen, collagen fibrils and/or collagen fibers produced by the methods of the present invention is suitable for a variety of uses. According to certain currently preferred embodiments, the collagen fibers produced by the methods of the invention are used for the production of textiles, particularly leather-like textiles. The natural, cell-free collagen fibers of the invention are suitable for use in a variety of textile-production methods, as are currently known and as will be known in the art.

According to some embodiments, the soluble collagen, collagen fibrils and/or collagen fibers produced by the methods of the present invention is suitable for medical and/or cosmetic uses.

According to some embodiments, the collagen is used as a skin filler. Fillers that contain collagen can be used cosmetically to remove lines and wrinkles from the face and can also improve scars.

According to some embodiments, the collagen is used in wound dressing.

According to some embodiments, the collagen is used in guided tissue regeneration. Collagen-based membranes can be used in periodontal and implant therapy to promote the growth of specific types of cells.

According to an aspect the present invention provides a system for producing soluble-collagen enriched medium, comprising:
(i) a first reservoir comprising collage-producing animal cells in a culture medium under conditions inducing collagen synthesis, the reservoir has an inlet and outlet; and
(ii) a delivery means configured to control delivery of a medium into and/or out of the first reservoir.

According to some embodiments, the delivery means further comprises a pump configured to deliver the medium into and/or out of the reservoir.

According to some embodiments, the delivery is continuous or intermittent.

According to some embodiments, the system further comprises a medium reservoir. According to certain embodiments, the pump is configured for pumping the medium from the medium reservoir to the first reservoir.

According to some embodiments, the system comprises a collecting reservoir.

According to certain embodiments, the medium delivered into the first reservoir is a fresh culture medium. According to some embodiments, the medium delivered out of the first reservoir is a soluble collagen enriched medium.

According to some embodiments, the system comprises means for monitoring medium/cells conditions

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1: Cell Culturing

1. 10⁶ bovine pluripotent cells were expanded under conditions supporting their pluripotency (International Patent Application Publication No. WO 2020/230138 to the Applicant of the present invention). The pluripotent stem cells, typically in aggregate form, were then induced to differentiate to stromal cells using 10 μM CHIR and 10 ng/ml Activin A for 4 days. These cells secreted soluble collagen into the growth medium.
2. Bovine embryonic fibroblast cells were expanded in standard media (DMEM+10% Fetal Bovine Serum+1% penicillin\streptomycin). 24 hours after seeding the medium was changed to basal media to increase the secretion of soluble collagen.

### Example 2: Collagen Extraction and Quantification

### Secreted Soluble Collagen

35,000 bovine embryonic fibroblast cells/cm² were seeded in standard medium. 24 hours later the medium was changed to basal medium and the soluble collagen was measured on day 6 or 7. Likewise, 20,000 stromal cells/cm² were seeded in basal medium. 24 hours later the cells were transfected with siRNA targeted to LOX or a scramble sequence as a control. After 24 hours the medium was changed to fresh basal medium. The soluble collagen levels were measured daily from day 3 until day 7.

Secreted soluble collagen was measured using QuickZyme soluble collagen assay which is based on binding of the dye Sirius Red to collagen. Following binding of the dye, the collagen-dye complex precipitates, resulting in a colored pellet. The pellet is re-suspended in an alkaline solution and the absorbance is measured in a plate reader.

### Sample Preparation

1. Conditioned culture medium is collected and centrifuged for 10 minutes at 1,500 g (at 4° C.) to remove cell debris.
2. The supernatant is diluted in a dilution buffer placed in the assay microplate (V-shaped plate).
3. The collagen standard solution is diluted in serial dilutions in the dilution buffer and placed in the assay microplate (V-shaped plate).
4. Sirius red dye is added to the samples and to the standards and mixed.
5. The plate is centrifuged for 60 minutes at 3000×g at 4° C. to precipitate the pellet.
6. The pellets are washed and resuspended.

The resuspended colored solution is transferred to the reading microplate (flat bottom) and read at 540 nm.

### Results

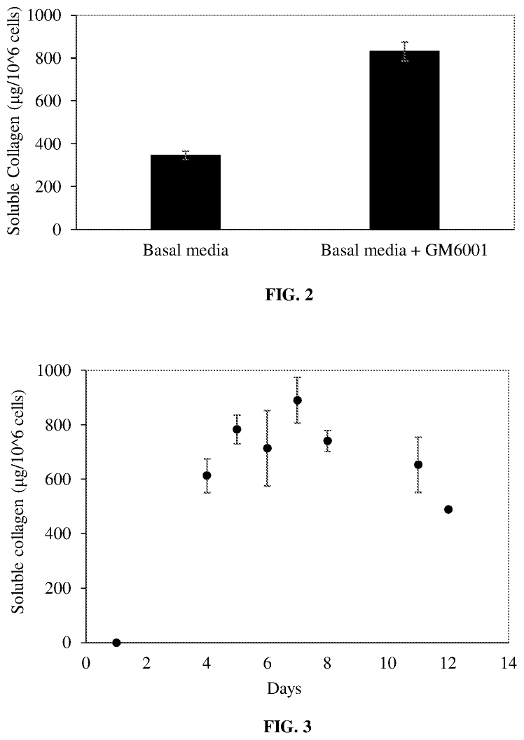

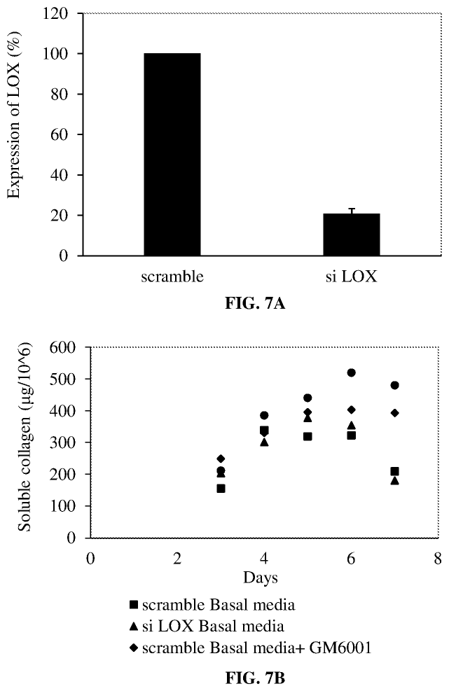

About 400 μg soluble collagen was measured for 10⁶ bovine embryonic fibroblasts (FIG. 2) and 10⁶ bovine stromal cells (FIG. 7B, scramble sequence).

### Insoluble Collagen

35,000 bovine embryonic fibroblast cells/cm2 were seeded in standard medium (DMEM+10% Fetal Bovine Serum+1% penicillin\streptomycin). 24 hours after seeding the medium was changed to basal medium with different concentration (0-500 μM) of GM6001, a matrix metalloprotease (MMP) inhibitor which prevents C-terminal cleavage of the procollagen. After 4 days insoluble collagen was collected from the culture dish and measured using “Total Collagen Assay kit” (QuickZyme Biosciences). This kit enables quantitative measurement of all types of collagen. The kit is based on the quantitative colorimetric determination of hydroxyproline residues, obtained by acid hydrolysis of collagen.

### Sample Preparation

1. Wash the cells once with PBS. Add 0.2 ml of PBS to one well (10 cm²) and scrape the cells using cell scraper, collect into screw-capped tubes (supplied with the kit).
2. Dilute 1:1 (v/v) with 12M HCl (final concentration 6M HCl), and incubate for 20 hours at 95° C. in a thermoblock.
3. After the incubation, allow the tubes to cool to room temperature, centrifuge the tubes for 10 min at 13,000 g. The supernatant is used for further analysis. Dilute the hydrolyzed sample with cell culture grade water as following: 1 volume sample+0.5 volume water (e.g., 200 μl hydrolysate+100 μl water). The sample will then be in 4M HCl. All further dilutions that might be required should be performed using 4M HCl. 35 μl of the diluted hydrolyzed sample is used for analysis in the assay.

### Quantification

1. Pipette 35 μl of the hydrolyzed samples (diluted to 4M HCl) into the appropriate wells of a 96-well plate. Add 75 μl assay buffer to each well and mix well, cover the plate with an adhesive plate seal and incubate for 20 minutes at room temperature, shake the plate every 5 min.
2. Prepare detection reagent (75 μl/well) by mixing detection reagents A and B at a ratio of 2:3 (30 μl+45 μl/well respectively).
3. Carefully remove the plate seal and add 75 μl detection reagent to each well. Cover the plate with an adhesive plate seal. Mix well by shaking the plate and incubate for 60 minutes at 60° C. in a plate-dry bath.
4. Cool the plate to room temperature on ice for no more than 5 minutes, carefully remove the plate seal and wipe dry the bottom of the plate.
5. Read the plate at 570 nm and analyze the data; calculate total collagen in the sample based on the standard curve measured values.

### Results

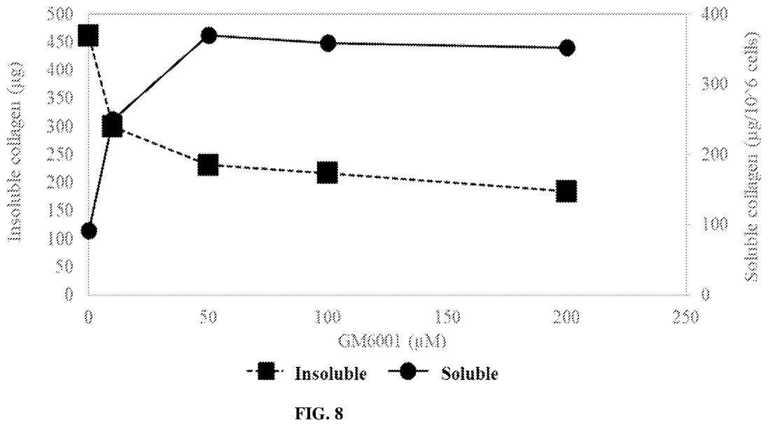

About 500 μg insoluble collagen was measured for 35,000 bovine embryonic fibroblasts/cm²; the amount is decreasing as the concentration of GM6001 is increasing (FIG. 8). As can be further taken from FIG. 8, decrease in the amount of insoluble collagen was accompanied by an increase in the amount of soluble collagen when the GM6001 concentration is increased up to about 50 μM.

### Example 3: Continuous Collection of Soluble Collagen from Bovine Embryonic Fibroblast Cells

35,000 bovine embryonic fibroblast cells/cm² were expanded in standard medium (DMEM+10% Fetal Bovine Serum+1% penicillin\streptomycin). 24 hours after seeding their medium was changed to basal medium with 50 μM GM6001. Soluble collagen levels were measured daily and reached maximum levels of ˜900 μg\10⁶ cells on the 7^{th} day. The culture stayed stable for 12 days (FIG. 3).

Next, 35,000 bovine embryonic fibroblast cells/cm² were seeded in standard medium (DMEM+10% Fetal Bovine Serum+1% penicillin/streptomycin). 24 hours after seeding the medium was changed to basal medium. After 4 days, the medium was collected and soluble collagen content was measured. Fresh basal medium applied to the cells. After 4 days the medium was collected and soluble collagen levels were measured.

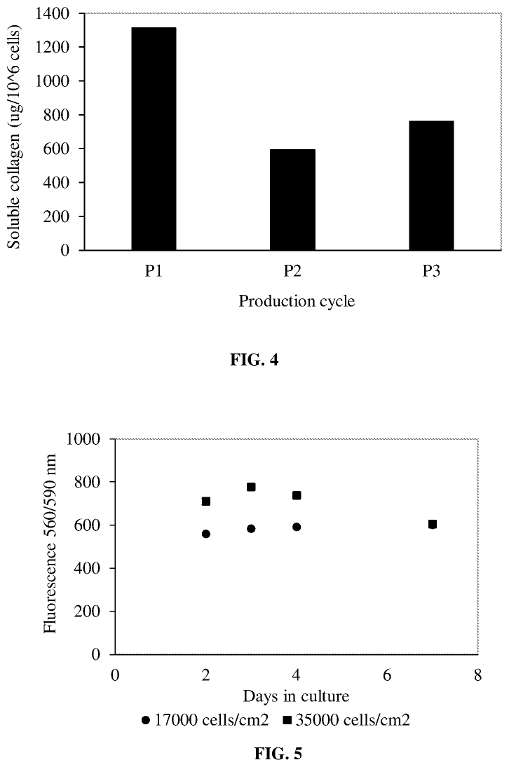

In addition, 35,000 bovine embryonic fibroblast cells/cm² were seeded in standard medium (DMEM+10% Fetal Bovine Serum+1% penicillin\streptomycin). 24 hours after seeding the medium was changed to basal medium. After 6 days medium was collected and soluble collagen content was measured. Cells were trypsinized and reseeded (35,000 bovine embryonic fibroblast cells/cm²) in standard medium (DMEM+10% Fetal Bovine Serum+1% penicillin\streptomycin). 24 hours after reseeding the medium was changed to basal medium. After 7 days (13 days total) the medium was collected and soluble collagen content was measured (P2). After additional 7 days (20 days total) the medium was collected and soluble collagen content was measured (P3) (FIG. 4).

### Example 4: Gene Expression Analysis of Col1A1, Col3A1 and Elastin Genes

The expression levels of bovine Collagen type 1, type 3, and Elastin (Col1A1, NM_001034039.2; Col3A1, NM_001076831.1; and Elastin NM_175772.2) was examined by Real time Quantitative PCR. 20,000 cells/cm² of bPSC were seeded and differentiated to stromal cells for 4 days. After 4 days the cells were passaged (P2) and lysed for RNA extraction at days 8, 14, 18 and 24. At day 14 the cells were passaged again (P3) and lysed for RNA extraction at days 18, 24, 28 and 35. The RNA was reverse transcribed to cDNA and 5 ng/μl were subjected to RT-PCR with the following primers:

β-Actin:
F:
(SEQ ID NO: 3
CACCACACCTTCTACAACG;
R:
(SEQ ID NO: 4)
TGTTGAAGGTCTCGAACATGA
Col1A1:
F:
(SEQ ID NO: 7
GGACACAGAGGTTTCAGTG;
R:
(SEQ ID NO: 8)
TCTCTCACCAGGCAGAC
Co13A1:
F:
(SEQ ID NO: 9)
CCTGAAATCCCGTTTGGAGA;
R:
(SEQ ID NO: 10)
CCTTGAGGTCCTTGACCATTAG
Elastin:
F:
(SEQ ID NO: 11)
TGCAGTGGTGCCTCAACTT;
R:
(SEQ ID NO: 12)
TGCGCCTGGAAGCACTC

### Results

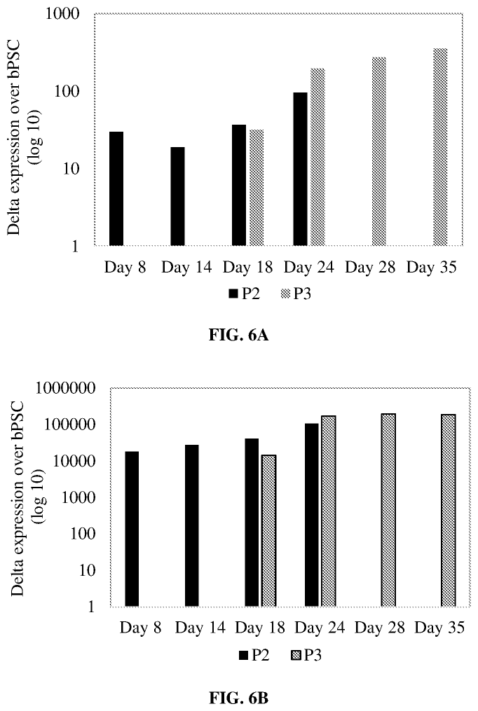

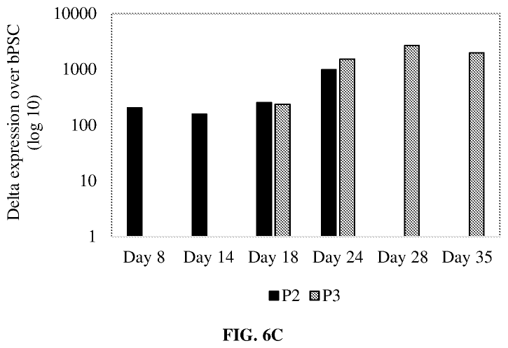

FIG. 6 demonstrates that as compared to undifferentiated pluripotent cells, the differentiated cells show significant increase in both Collagen type 1 and type 3, and also in Elastin, already after 8 days, which is comparable to the expression level in fibroblasts. β-Actin was used as endogenous control to compare the RNA amounts of the samples.

### Example 5: Enhancement of Soluble Collagen by Inhibition of Procollagen N- and C-terminals

Basal medium supplement with GM6001, matrix metalloprotease (MMP) inhibitor which inhibits C-terminal cleavage of the procollagen, enhanced the secreted soluble collagen in the growth medium of bovine stromal cells and bovine embryonic fibroblasts.

### Results

Addition of 50 μM GM6001 enhanced the amount of secreted soluble collagen to about 1 mg soluble collagen for 10⁶ bovine embryonic fibroblasts (FIGS. 2 and 3).

Silencing of LOX together with addition of 50 μM GM6001 enhanced the amount of soluble collagen in the growth medium of bovine stromal cells as can be seen in FIG. 7B.

### Example 6: Gene Silencing Using siRNA

The Bovine LOX (lysyl oxidase) gene (Gene ID: 280841) was knockdown using siRNA. 20,000 bovine stromal cells/cm² were transfected with 20 nM of siRNA sequence against LOX (5′GCUGAUAACCAGACGGCACUU3′; SEQ ID NO:1) or a scramble sequence (5′UGGUUUACAUGUCGACUAAUU3′; SEQ ID NO:2) as control, using Dharmacon DharmaFECT 1 transfection reagent. 24 hours after transfection the cells were lysed for RNA extraction. The RNA was reverse transcribed to cDNA and 5 ng/μl were subjected to RT-PCR with the following primers:

β-Actin:
F:
(SEQ ID NO: 3)
CACCACACCTTCTACAACG;
R:
(SEQ ID NO: 4)
TGTTGAAGGTCTCGAACATGA.
LOX:
F:
(SEQ ID NO: 5)
AGGGTGCTGCTAAGATTTCC;
R:
(SEQ ID NO: 6)
GTCACACGATGTGTCCTCAA.

### Results

RT-PCR analysis of LOX mRNA level showed an 80% reduction of LOX expression by siRNA sequence, relative to the control (FIG. 7A).

### Example 7: Cell Proliferation Measurement

Cell proliferation was assessed using PrestoBlue reagent. 35,000 cells/cm² or 17,000 cells/cm² of bovine embryonic fibroblasts were seeded in a 6-well plate with standard medium. 24 hours later the medium was replaced to a fresh basal medium and 24 hours later presto blue was added to the medium for 3 hours. 120 μl of this medium were taken into 96 well black plate and fluorescence was measured: Ex 560 nm and Em 590 nm. The measurement was repeated every day in a new well for 7 days.

### Results

Bovine embryonic fibroblasts stimulated by basal medium to enhance soluble collagen become non-proliferative cells as can be seen in FIG. 5.

### Example 8: GM6001 Increases Production of Soluble Collagen While Reducing Insoluble Collagen Levels

35,000 bovine embryonic fibroblast cells/cm² were seeded in standard medium (DMEM+10% Fetal Bovine Serum+1% penicillin\streptomycin). 24 hours after seeding the medium was changed to basal medium with different concentration (0-500 μM) of GM6001, a matrix metalloprotease (MMP) inhibitor which prevents C-terminal cleavage of the procollagen. After 4 days, the medium was collected from the cultures and measured for soluble collagen levels. Insoluble collagen was collected from the culture dish its level was measured. As can be seen in FIG. 8, GM6001 affect the concentration of both soluble and insoluble collagen: soluble collagen levels increased in correlation with the decrease in the insoluble collagen levels.

### Example 9: Enrichment of Growth Media with Soluble Collagen by Continues Production Process

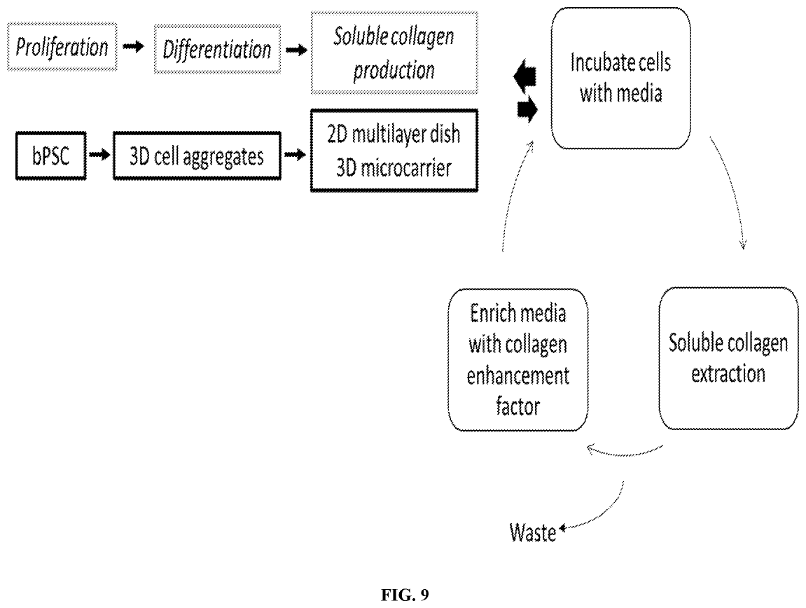

FIG. 9 is a schematic presentation of continuous soluble collagen production. Bovine pluripotent cells are proliferated and seeded to form 3D aggregates. Differentiation medium applied to the cell aggregates, direct the cells to collagen producing committed cells. Aggregates of the collagen producing committed cells are seeded on 2D multilayer system or 3D microcarrier allowing the cells to complete the differentiation. When culture is in full confluency, soluble collagen production medium (basal media with GM6001) is applied. Next, soluble collagen enriched growth media is collected, waste and side products are removed, and ECM components are separated from the medium. Then, nutrients and soluble collagen enhancement factors are added to the growth media that is applied again to the cells.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-67. (canceled)

68. A method of producing cell-free animal collagen comprising the steps of:
a. growing a plurality of collagen producing animal cells in a cell culture comprising culture medium under conditions inducing collagen synthesis, wherein soluble collagen comprising procollagen is secreted from the plurality of collagen producing animal cells to the culture medium;
b. inhibiting or reducing the cleavage of at least one propeptide from the procollagen N-terminus, the procollagen C-terminus or from a combination thereof, thereby obtaining a soluble collagen-enriched medium; and
c. collecting the soluble collagen enriched medium while maintaining the cell culture.

69. The method of claim 68, wherein the conditions inducing collagen synthesis comprise growing the cells in a culture medium comprising at least one agent that induces collagen synthesis selected from the group consisting of: iron, insulin, ascorbic acid or a salt thereof, and any combination thereof.

70. The method of claim 68, wherein inhibiting or reducing the cleavage of the at least one propeptide from the procollagen N- or C-terminus comprises supplementing the medium with at least one agent selected from: a small molecule, an antibody, a protein, a peptide and an siRNA.

71. The method of claim 70, wherein the small molecule, antibody, protein, peptide or siRNA inhibits or reduces the activity of at least one procollagen peptidase.

72. The method of claim 71, wherein the small molecule, antibody, protein, peptide or siRNA is a matrix metalloprotease (MMP) inhibitor.

73. The method of claim 68, wherein collecting the soluble collagen enriched medium is performed by continuously or intermittently removing the soluble collagen enriched medium.

74. The method of claim 68, wherein the method further comprises furnishing the cell culture with a collagen-free culture medium comprising at least one agent inhibiting the cleavage of at least one propeptide from procollagen N- or C-terminus and at least one agent inducing collagen synthesis by the cells.

75. The method of claim 74, wherein the collagen-free culture medium further comprises at least one agent inhibiting crosslinking of soluble collagen secreted to the medium.

76. The method of claim 68, wherein at least one of the cells are genetically modified to:
(1) enhance collagen synthesis, enhance collagen secretion, inhibit collagen crosslinking, or a combination thereof; and/or
(2) inhibit or reduce the cleavage of at least one propeptide from the procollagen N- or C-terminus; and/or
(3) reduce activity and/or expression of at least one procollagen peptidase; and/or
(4) reduce the expression and/or activity of at least one Lysyl oxidase (LOX); and/or
(5) enhance expression and/or activity of at least one protein selected from the group consisting of COL1A1, COL1A2, COL3A1, and any combination thereof; and/or
(6) increase expression and/or activity of lysyl hydroxylase (LH), Prolyl hydroxylase (PH), or a combination thereof; and/or
(7) alter expression of at least one gene involved in the feedback inhibition of collagen synthesis.

77. The method of claim 68, wherein the method further comprises one or more of the following steps:
(1) genetically modifying the cells to increase collagen production;
(2) genetically modifying the cells to increase collagen secretion;
(3) genetically modifying the cells to reduce the cleavage of a propeptide from the procollagen N- or C-terminus; and/or
(4) genetically modifying the cells to reduce collagen crosslinking.

78. The method of claim 68, wherein the cells are non-genetically modified cells.

79. The method of claim 68, wherein the method further comprises a step of inhibiting crosslinking of the soluble collagen within the soluble collagen-enriched culture medium.

80. The method of claim 79, wherein inhibiting crosslinking of the soluble collagen comprises one or more of the following steps:
(1) depleting copper ions from the culture medium;
(2) replacing the medium with a cooper-free medium; and
(3) supplementing the medium with at least one lysyl oxidase inhibitor.

81. The method of claim 68, wherein the cell culture is maintained for a production cycle of at least 5 days.

82. The method of claim 68, wherein the method results in cell-free animal soluble collagen production of at least 100, at least 200, at least 300, at least 400, at least 500 or at least 600 μg/10⁶ cells per production cycle.

83. The method of claim 68, wherein the soluble-collagen enriched medium further comprises at least one additional ECM component.

84. The method of claim 68, the method further comprises subjecting the soluble collagen in the collected soluble-collagen enriched medium or a soluble collagen obtained therefrom to (1) conditions enabling the soluble collagen assembly to collagen fibrils; or (2) conditions enabling the soluble collagen assembly to collagen fibrils and crosslinking the collagen fibrils to form collagen fibers.

85. A soluble collagen produced by the method of claim 68.

86. A collagen fibril or collagen fiber produced by the method of claim 84, or a medium comprising the same.

87. A composition comprising culture medium and soluble collagen, wherein the soluble collagen comprises at least 50% procollagen.

88. The composition of claim 87, wherein the composition is devoid of hydrolyzed collagen.

89. A textile, food product, cosmetic product or medical material comprising at least one of the soluble collagens of claim 85.

90. A textile, food product, cosmetic product or medical material comprising the composition of claim 87.

91. A system for producing soluble collagen-enriched medium, comprising:
a. a first reservoir comprising collage-producing animal cells in a culture medium under conditions inducing collagen synthesis, the reservoir has an inlet and outlet; and
b. delivery means for controlling the culture medium composition.


SEQUENCE LISTING (12 sequences)

SEQ ID NO: 1 - RNA, 21 nt, Artificial sequence
  siRNA
gcugauaaccagacggcacuu

SEQ ID NO: 2 - RNA, 21 nt, Artificial sequence
  siRNA
ugguuuacaugucgacuaauu

SEQ ID NO: 3 - DNA, 19 bp, Artificial sequence
  primer
caccacaccttctacaacg

SEQ ID NO: 4 - DNA, 21 bp, Artificial sequence
  Primer
tgttgaaggtctcgaacatga

SEQ ID NO: 5 - DNA, 20 bp, Artificial sequence
  Primer
agggtgctgctaagatttcc

SEQ ID NO: 6 - DNA, 20 bp, Artificial sequence
  Primer
gtcacacgatgtgtcctcaa

SEQ ID NO: 7 - DNA, 19 bp, Artificial sequence
  Primer
ggacacagaggtttcagtg

SEQ ID NO: 8 - DNA, 17 bp, Artificial sequence
  Primer
tctctcaccaggcagac

SEQ ID NO: 9 - DNA, 20 bp, Artificial sequence
  Primer
cctgaaatcccgtttggaga

SEQ ID NO: 10 - DNA, 22 bp, Artificial sequence
  Primer
ccttgaggtccttgaccattag

SEQ ID NO: 11 - DNA, 19 bp, Artificial sequence
  Primer
tgcagtggtgcctcaactt

SEQ ID NO: 12 - DNA, 17 bp, Artificial sequence
  Primer
tgcgcctggaagcactc